(19) United States
(12) Patent Application Publication
(10) Pub. No.: US 2014/0356394 A1
(43) Pub. Date: Dec. 4, 2014

(54) COMPOSITION FOR TREATMENT OR PREVENTION OF OBESITY, CONTAINING WATER EXTRACTS OF FOMITELLA FRAXINEA

(71) Applicants: POSTECH ACADEMY-INDUSTRY FOUNDATION, Gyungbuk (KR); KYEONGSANGBUK-DO, Daegu (KR)

(72) Inventors: Sung Ho Ryu, Gyungbuk (KR); Hyeongji Lee, Busan (KR); Taehoon Lee, Seoul (KR); Jaeyoon Kim, Gyungbuk (KR); Jong-Vong Eun, Daegu (KR); Kang-Ha Kim, Gyungbuk (KR); Sang-Gap Shim, Gyungbuk (KR); Woo-Jae Cheon, Gyungbuk (KR)

(21) Appl. No.: 14/363,899

(22) PCT Filed: Dec. 7, 2012
(86) PCT No.: PCT/KR2012/010621

§ 371 Date: Jun. 9, 2014

(30) Foreign Application Priority Data
Dec. 9, 2011 (KR) 10-2011-0131904

(51) Int. Cl. A61K 36/06; A23L 2/52; A23L 1/30

(52) U.S. Cl. CPC A61K 36/06; A23L 1/3002; A23L 2/52
USPC 424/195.15

(57) ABSTRACT

The present invention relates to a composition for the treatment or prevention of obesity, the composition containing water extracts of Fomitella fraxinea as an active ingredient, and to a medicine and a dietary supplement, containing the extracts.

### TECHNICAL FIELD

The present invention relates, in general, to an extract of Fomitella fraxinea (Fr.) Imaz. (hereinafter referred to as Fomitella fraxinea), and, more particularly, to a water extract of Fomitella fraxinea effective in treating and preventing obesity, a use thereof, and a method of obtaining the same.

### BACKGROUND ART

Obesity refers to the excessive accumulation of fat tissue in the body. Obesity is known as a main cause of cardiovascular disease, type 2 diabetes, and cancer, for example, colon cancer, rectal cancer, prostate cancer, breast cancer, ovarian cancer, etc. (Haslam D W, James W P, 2005, “Obesity”. Lancet 366 (9492): 1197-209). A population of obesity in Korea increases steadily, and worldwide childhood obesity has tripled in 10 years. Rapidly growing population of adult obesity is expected. Many reports have been proposed for treatment and prevention of obesity (Lau D C, Douketis J D, Morrison K M, Hramiak I M, Sharma A M, Ur E (April 2007), “2006 Canadian clinical practice guidelines on the management and prevention of obesity in adults and children [summary]”. CMAJ 176 (8): S1-13., Wada K, Ikeda Y.: Longitudinal studies to determine the effect of body fat rate reduction on blood pressure. J Med Sys 22: 19-25 (1998)).

The biggest cause of obesity is excess calories in the diet and lack of exercise. As a result, excess energy is accumulated in the form of body fat (Bray G A, Popkin B M.: Dietary fat intake dose affect obesity. Am. J. Clin. Nutr 68: 1157-1173 (1998)). Currently, research has been actively conducted globally to develop an anti-obesity effective material. Accordingly, there is a growing interest in the anti-obesity efficacy evaluation model.

Current drug treatment of obesity split into two main types: first type drugs which act directly on the nervous system, the transmission system or the hormones to suppress appetite or make feel satiety, such as, appetite suppressant, energy metabolism accelerators, and regulators for peptide or hormones, and the second type drugs which act indirectly on the digestive enzymes to suppress the absorption.

On the other hand, drugs which act directly on the nervous system, the transmission system or the hormone to suppress appetite or satiety might occur side effects such as rise of blood pressure medications, insomnia, sweating, lethargy, etc. Because of these problems, in recent years, lipid absorption inhibitor which acts on the digestive enzymes to inhibit the absorption of lipid has been focused. Xenical™ (orlistat) is well known as drugs with the mechanism which suppresses activity of lipolytic enzyme by forming a covalent bond with serine which is the active site of gastrointestinal lipase (Triglyceride lipase, TGL). Lipid absorption inhibitor only act locally and systemic absorption is negligible, so there is no central nervous system side effects. Because of these benefits, the market of the obesity drug to inhibit lipid absorption has shown steady growth each year.

Meanwhile, there are some 10,000 different mushrooms reported in the world. Due to their high value as edible foods and pharmaceutical uses, a considerable amount of research has been conducted in Europe, the U.S., Japan, etc., so as to secure them as beneficial microorganism resources. More specifically, according to research results, the physiologically active materials produced by mushrooms have few adverse effects and are thus safe in terms of toxicity. Additionally, they serve various functions, such as regulating functions within the human immune system, providing anti-cancer effects, regulation of metabolism, etc.

The inventors of the present invention, after prolonged research efforts into the development of natural substances having improved effects on obesity by inhibiting lipid absorption in the body, have successfully discovered such substances not having any adverse effects in vivo, thus realizing the present invention.

### DISCLOSURE

### Technical Problem

The present invention aims to provide a composition effective for treating or preventing obesity a by inhibiting lipid and cholesterol absorption in the body without causing any adverse effects in the body, a pharmaceutical drug, and a functional health food containing the same.

### TECHNICAL SOLUTION

In accordance with an aspect thereof, the present invention provides a composition containing a water extract of Fomitella fraxinea as an active ingredient for treating or preventing obesity.

Additionally, the present invention provides a pharmaceutical drug containing a water extract of Fomitella fraxinea as an active ingredient for treating or preventing obesity.

Furthermore, the present invention provides a method for treating or preventing obesity by administering a pharmaceutically effective amount of a water extract of Fomitella fraxinea to a patient.

Still further, the present invention provides a functional health food containing a water extract of Fomitella fraxinea as an active ingredient for improving or preventing obesity.

In an exemplary embodiment of the present invention, there is provided a composition containing a water extract of Fomitella fraxinea as an active ingredient for treating or preventing obesity.

Fomitella fraxinea, Japan name Beth Kou Takeo, belongs to the Aphyllophorales, Polyporaceae and grows from spring to autumn round in the dead broadleaf trees or root of living trees. Fomitella fraxinea is a one year or perennial mushroom. Fomitella fraxinea is distributed in Korea, Japan, Europe, and America and is known as medicinal mushroom with anticancer and immune booster properties.

The inventors of the present invention have studied natural substances which act on the digestive enzymes to inhibit lipid absorption to replace Xenical™ (orlistat). The inventors have founded that extract of Fomitella fraxinea, in particular, water extract of Fomitella fraxinea effectively inhibit the activity of gastrointestinal lipase (triglyceride lipase, TGL). In addition, the extract inhibits the absorption of cholesterol into the blood. The extract is effective for the treatment and prevention of obesity, and also is effective for the treatment and prevention of hyperlipidemia.

In particular, the water extract of Fomitella fraxinea may be prepared according to a conventional method of plant extraction. In a preferable embodiment, Fomitella fraxinea, is dried and pulverized, followed by extraction by adding the resulting powder in an amount of 0.1 g to 20 g and preferably in an amount of 1 to 5 g to 100 mL of water. When the amount of the pulverized resultant is too little relative to the volume of the extraction solvent, the effect of Fomitella fraxinea will not be sufficient and is thus undesirable. In contrast, when the amount of the pulverized resultant is in excess relative to the volume of the extraction solvent, there is no significant level of increase in the effect of Fomitella fraxinea, but results in an increase in production cost and is thus not desirable in terms of productivity.

In this regard, there is no particular limitation on the extraction conditions. Preferably, however, a water extract of Fomitella fraxinea may be obtained by mixing Fomitella fraxinea with water as an extraction solvent at a temperature of from 20 to 60° C. for from 12 to 36 hours, more preferably at from 30 to 40° C. for from 20 to 24 hours.

It takes a long time to extract the active ingredients at too low of a temperature. On the other hand, too high of an extraction temperature deteriorates the activities of the active ingredients. Furthermore, when the extraction is performed for too short of a time, an insufficient concentration of the active ingredients is obtained; whereas, when too long of an extraction time is undesirable in terms of productivity because the increment of the concentration of the extract is negligible compared to the extended time of extraction.

The water extract of Fomitella fraxinea thus obtained may be filtrated using a filter cloth or the like, and after the centrifugation of the filtrate, the resulting pellet may be removed whereas the supernatant may be concentrated to dryness at reduced or normal pressure before lyophilization.

Although no particular limitation is imparted thereto, the water extract of Fomitella fraxinea as an active ingredient is preferably in the range of from 0.001 to 50 wt % of the composition for treating or preventing obesity according to an exemplary embodiment of the present invention. When the content of the water extract of Fomitella fraxinea as an active ingredient is less than 0.001 wt %, the effect of inhibiting absorption of lipid may be negligible; whereas a content exceeding 50 wt % is economically disadvantageous because a further increase in the content produces negligible effects. The composition more preferably contains the water extract of Fomitella fraxinea in an amount of from 0.01 to 50 wt % and further more preferably in an amount of from 0.1 to 30 wt %.

In an exemplary embodiment of the present invention, there is provided a pharmaceutical drug containing a water extract of Fomitella fraxinea as an active ingredient included in the composition. The pharmaceutical drug containing the water extract of Fomitella fraxinea as an active ingredient may further include a carrier, an excipient, and diluents, as appropriate.

Examples of carriers, excipients, and diluents to be included in the pharmaceutical drug containing the water extract of Fomitella fraxinea of the present invention as an active ingredient may include: lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and a mineral oil.

The pharmaceutical drug containing the water extract of Fomitella fraxinea of the present invention as an active ingredient may be formulated into oral dosage forms such as powders, granules, tablets, suspensions, emulsions, syrups, etc., according to a conventional method, respectively.

The oral dosage formulations may include solid formulations and liquid formulations. Examples of the solid formulations for oral administration may include pills, powders, granules, capsules, etc. The solid formulations may include at least one excipient in addition to the water extract, for example, starch, calcium carbonate, sucrose or lactose, gelatin, etc. In addition to the simple excipient, a lubricant such as magnesium stearate and talc may also be added. Examples of the liquid formulations for oral administration may include suspensions, liquid medicines for internal use, emulsions, syrups, etc. In addition to the simple diluent such as water and liquid paraffin, various excipients such as humectants, sweeteners, fragrants, and preservatives may also be used.

A preferred dosage of the composition of the present invention containing a water extract of Fomitella fraxinea may vary depending on the individual subject's health conditions, body weight, severity of illness, formulation types, administration routes, etc., but may be appropriately chosen by one with ordinary skill in the art. Preferably, the daily dosage of the pharmaceutical drug of the present invention containing the water extract of Fomitella fraxinea may be between 0.0001 to 100 mg/kg relative to the amount of the water extract of Fomitella fraxinea, and more preferably, between 0.01 to 10 mg/kg. The pharmaceutical drug may be administered once or a few times daily. Nevertheless, the above dosage and the frequency of administration should not be construed as limiting the scope of the present invention in any manner.

In another exemplary embodiment of the present invention, there is provided a health functional food containing a water extract of Fomitella fraxinea as an active ingredient for improving or preventing obesity. The term “functional health food” used herein refers to a natural or processed product containing at least one nutrient, preferably one which has become readily edible by having undergone a certain level of processing.

Examples of the functional health food to add the water extract of Fomitella fraxinea of the present invention may include various kinds of foods, beverages, gums, teas, vitamin complexes, etc. In addition, the foods of the present invention may include special nutrient foods (e.g., milk formulas, infants formulas, etc.), processed meat products, fish products, tofus, starch jellies, noodles (e.g., ramens, thin noodles, etc.), health supplement foods, seasoned foods (e.g., soy sauce, bean paste, red pepper paste, a mixed soy paste, etc.), sauces, confectioneries (e.g., snacks), milk products (e.g., fermented milks, cheeses, etc.), pickled foods (e.g., various kinds of kimchis, pickled vegetables, etc.), beverages (e.g., fruit juices, vegetable juices, soy milks, fermented beverages, etc.), and natural seasonings (e.g., ramen seasonings, etc.), but are not limited thereto. The above foods, beverages, or food additives may be manufactured according to a conventional manufacturing method.

The term, a functional food used herein, refers to a processed food designed to help regulate the body's natural biorhythms, prevent diseases or help a person recover from diseases, etc. Such foods are rendered with added value by means of physical, biochemical, and bioengineering technologies upon the group of foods or food compositions so that they can act and express the functions of a given food for a particular purpose, within live subjects. The functional food may further include a sitologically acceptable food supplement additive, and may further include other additives as appropriate, such as a carrier, an excipient, and a diluent, which are commonly used in the manufacture of functional foods.

The term, “beverage” used herein, is a collective term referring to all the drinks to be taken so as to quench thirst or enjoy the taste. The “beverage” should include as an active ingredient a water extract of Fomitella fraxinea in a predetermined ratio but is not particularly limited regarding other ingredients. The beverage may further include various flavors or natural carbohydrates, etc., as is the case with conventional drinks. Examples of natural carbohydrates may include conventional sugars such as: monosaccharides, e.g., glucose, fructose, etc.; disaccharides, e.g., maltose, sucrose; and polysaccharides, e.g., dextrin, cyclodextrin, etc.; and a sugar alcohol such as xylitol, sorbitol, erythritol, etc. In addition to the sweeteners listed above, natural sweeteners such as thaumatin and stevia extracts (e.g., rebaudiocide A, glycyrrhizin, etc.), and other synthetic sweetenters (saccharin, aspartame, etc.) may be added. The natural carbohydrate may be added from about 1 to 20 g per 100 mL of a composition of the present invention, preferably from 5 to 12 g. Additionally, the composition of the present invention may further include the flesh of fruits for the manufacture of natural fruit juices, fruit juice beverages, and vegetable beverages.

Furthermore, the functional health food of the present invention may further include various nutrients, vitamins, minerals (electrolyte), natural and synthetic flavors, coloring agents, flavor enhancers (cheeses, chocolates, etc.), pectic acid and its salts, alginic acid and its salts, organic acids, protective colloid thickeners, pH adjusters, stabilizers, preservatives, glycerin, water, carbonating agents used in carbonated beverages, etc. The above ingredients may be used independently or in combination with others. Although the content of the additives may not be important, they may be used within the range of from 0 to 20 parts by weight per 100 parts by weight of the water extract of Fomitella fraxinea.

The term, “functional beverage” used herein, refers to a processed beverage designed to help regulate the body's natural biorhythms, prevent diseases or help a person recover from diseases, etc. Such functional beverages are rendered with an added value by means of physical, biochemical, and bioengineering technologies on a group of beverages or beverage compositions so that they can act and express the functions of a given food for a particular purpose, within live subjects.

The functional beverage should include as an active ingredient a water extract of Fomitella fraxinea in a predetermined ratio, but is not particularly limited regarding other ingredients. The functional beverage may further include various flavors or natural carbohydrates, etc., as is the case with conventional drinks. Examples of natural carbohydrates may include a conventional sugar such as monosaccharides, e.g., glucose, fructose, etc.; disaccharides, e.g., maltose, sucrose; and polysaccharides, e.g., dextrin, cyclodextrin, etc.; and a sugar alcohol such as xylitol, sorbitol, erythritol, etc. In addition to the sweeteners above, natural sweeteners, such as thaumatin and stevia extracts (e.g., rebaudiocide A, glycyrrhizin, etc.), and synthetic flavors (saccharin, aspartame, etc.) may be added. The natural carbohydrate may be added from about 1 to 20 g per 100 mL of a composition of the present invention, preferably from 5 to 12 g. Additionally, the composition of the present invention may further include flesh of fruits for the manufacture of natural fruit juices, fruit juice beverages, and vegetable beverages.

Additionally, the functional health food for improving or preventing obesity may contain the water extract from 0.01 to 15 wt % of the total food of the functional health food, and the beverage composition may contain the water extract from 0.02 to 5 g per 100 mL, preferably from 0.3 to 1 g.

In an exemplary embodiment of the present invention, there is provided a method for manufacturing the water extract of Fomitella fraxinea.

The method for manufacturing the water extract of Fomitella fraxinea may include preparing Fomitella fraxinea, mixing Fomitella fraxinea with water, and extracting the resulting mixture at a temperature of from 20 to 60° C. for from 12 to 36 hours. The details of the time, temperature, and mixed ratio of a solvent for the extraction of Fomitella fraxinea are the same as described above.

### Advantageous Effects

According to the present invention, the composition including a water extract of Fomitella fraxinea as an active ingredient can inhibit the absorption of neutral fat in the body and cholesterol, and is thus useful for treating or preventing obesity.

### DESCRIPTION OF DRAWINGS

FIG. 1 is a graph showing the comparative results of the inhibitory effect between the extracts of Fomitella fraxinea prepared in Example and Comparative Example upon TGL activity.

FIG. 2 is a graph showing the comparative results of the inhibitory effect between the extracts of Fomitella fraxinea prepared in Example and Comparative Example upon CEL activity.

FIG. 3 is a graph showing the results of the amount of lipid absorbed into blood from the digestive tract when a water extract of Fomitella fraxinea according to an exemplary embodiment of the present invention and orlistat, a conventional lipid inhibitor, were compared.

### MODE FOR INVENTION

A better understanding of the present invention regarding its invention features and effects may be obtained through the following examples which are set forth to illustrate, but are not to be construed as the limit of the present invention.

The details of the present invention are described infra.

### Example

### Preparation of a Water Extract of Fomitella fraxinea

A mixture of 5 g of a dry powder of Fomitella fraxinea (Fomitella fraxinea (Fr.) Imaz.) Hanabiotech Inc.) and 100 mL of water, as a solvent, was subjected to extraction at 37° C. for 24 hours in a shaking incubator. After centrifugation at 2500 rpm for 10 minutes, the supernatant was recovered as a water extract of Fomitella fraxinea.

### Comparative Example

### Preparation of an Ethanol Extract of Fomitella fraxinea

An ethanol extract of Fomitella fraxinea was prepared in the same manner as in Example except that 100 mL of ethanol (99.9%(v/v)) was used as an extraction solvent instead of water.

### Experimental Example

### 1. Measurement of the Activity of Pancreatic Lipase Upon Treatment with Fomitella fraxinea Extracts

Pancreatic lipase is known to non-specifically recognize substrates having an acyl chain and separate the acyl chain therefrom. The activities of pancreatic lipase in a group treated with the Fomitella fraxinea extracts prepared in the Example and the Comparative Example were measured using porcine pancreatic lipase Type 11 (Sigma Chemical).

The change in the activity of pancreatic lipase was observed by a color reaction considering that pancreatic lipase can decompose into p-nirophenylbutyrate, a chromogenic substrate. The inhibitory effects of the extracts upon pancreatic lipase were measured by a microplate reader based on the change in absorbance at 405 nm, and the optical density of a group not treated with the extracts was calculated in % with reference to the absorbance at 405 nm as a base value.

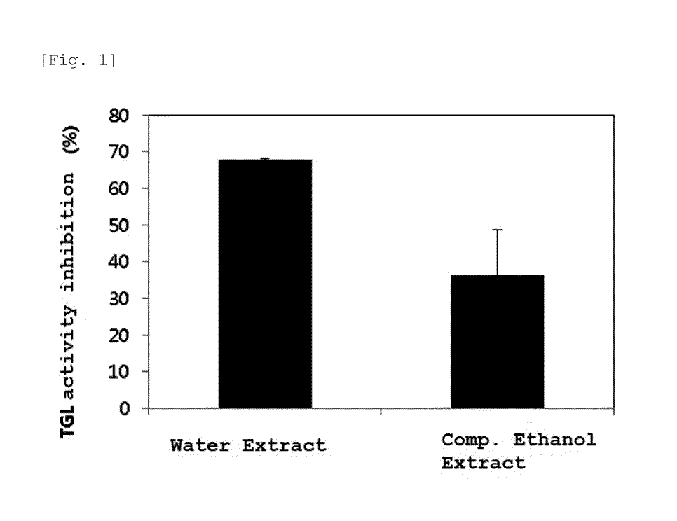

The changes in pancreatic lipase activity by the treatment with extracts prepared in the Example and the Comparative Example were measured. The results revealed that the water extract of Fomitella fraxinea prepared in the Example considerably decreased the activity of pancreatic lipase, whereas the ethanol extract of Fomitella fraxinea prepared in the Comparative Example showed almost no inhibitory effect against the activity of pancreatic lipase. The results are briefly shown in FIG. 1.

### 2. Measurement of the Activity of Pancreatic Cholesterol Esterase Upon Treatment with Fomitella fraxinea Extracts

Pancreatic cholesterol esterase is known to non-specifically recognize substrates having an acyl chain and separate the acyl chain therefrom. For use in measuring the effect of the Fomitella fraxinea extracts prepared in the Example and the Comparative Example on the activity thereof, porcine pancreatic cholesterol esterase was purchased from Sigma Chemical.

Pancreatic cholesterol esterase was measured of its activity change by a color reaction based on the enzymatic decomposition of a chromogenic substrate p-nitrophenylbutyrate. Absorbance at 405 nm was read on a microplate reader and used to express the inhibitory effects of the extracts on the pancreatic cholesterol esterase as a % of that treated with neither the ethanol extract nor the water extract.

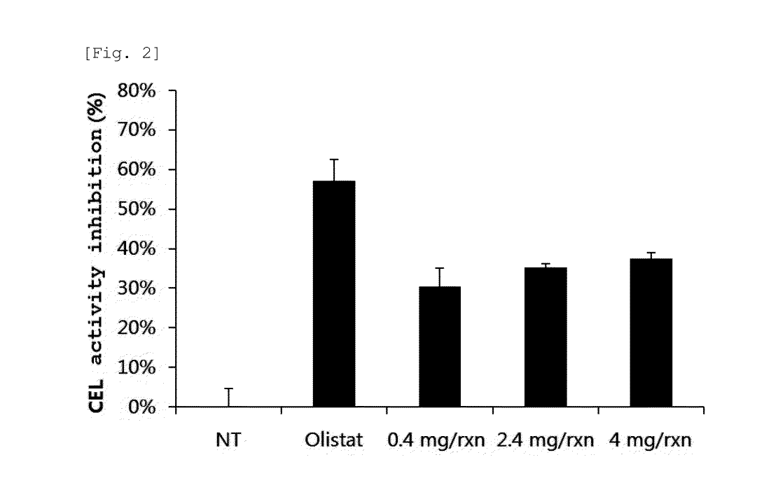

The changes in pancreatic cholesterol esterase activity by the treatment with extracts prepared in the Example and the Comparative Example were measured. The results revealed that the water extract of Fomitella fraxinea prepared in the Example considerably decreased the activity of pancreatic cholesterol esterase, whereas the ethanol extract of Fomitella fraxinea prepared in the Comparative Example showed almost no inhibitory effect against the activity of pancreatic cholesterol esterase. The results are briefly shown in FIG. 2.

### 3. Measurement of the Amount of Lipid Absorption Upon Treatment with Water Extract of Fomitella fraxinea

In order to confirm whether the water extract of Fomitella fraxinea prepared in the Example, which exhibited an inhibitory effect against the pancreatic lipase, also shows any inhibitory effect at the level of an individual subject, the lipid absorption in the digestive tract was measured.

Triolein (Amersham, 100 μCi/mL) radiolabeled with ¹⁴C was mixed with 1% (w/v) sodium carboxylmethyl cellulose (CMC-Na) and 1% (v/v) Tween 80, and then 0.1 mL of the mixture was forcibly administered into each mouse through an oral zonde so that 1 μCi could be delivered to each mouse.

Here, the mouse in the control group (an 8-week old male Balb/c with body weight of 25 g) was treated with 400 μg of Orlistat (20 mg/kg), which is used as an obesity treatment, and the mouse in the experimental group (an 8-week old male Balb/c with body weight of 25 g) was treated with 0.5 mL of the water extract (50 mg/mL) of Fomitella fraxinea prepared in the Example after concentrating it into 0.1 mL.

Six hours after the administration, blood samples were collected from the mice, and plasma was obtained by centrifuging the blood at 40° C. at the rate of 14000 rpm, and the radioactive dose was measured by a liquid scintillator (Beta counter, Beckman LS1801). The control group was not treated with anything. The amount of lipid absorption was determined as a relative value by dividing the radioactive dose of each group into the radioactive dose of the control group, in which the radioactive dose of the control group was set at ‘1’.

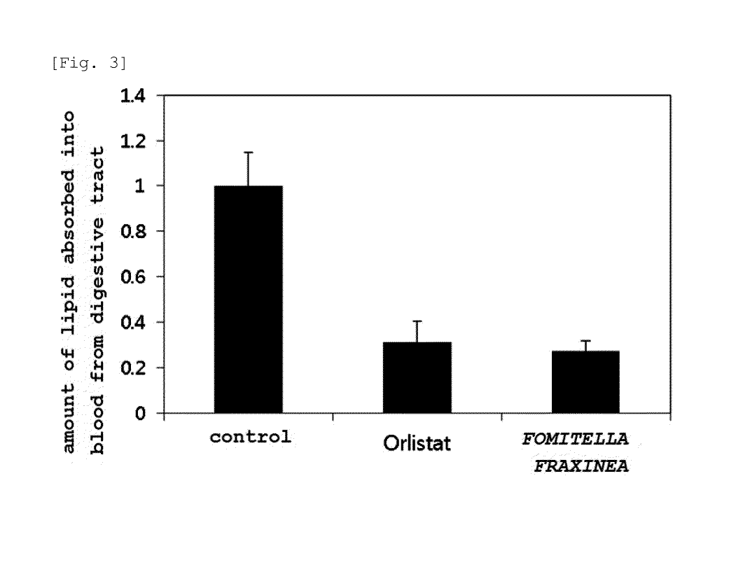

The water extract of Fomitella fraxinea of the present invention showed a significant inhibitory effect upon lipid absorption, as in the control group treated with Orlistat. The results are shown in FIG. 3.

In particular, the result on the control group in FIG. 3 was obtained from the blood sample of a mouse not treated with any lipid absorption inhibitor.

Examples of formulations manufactured using the water extract of Fomitella fraxinea are described herein below, but the formulations of the present invention are not limited thereto.

### Manufacturing Example

### Formulations Manufactured Using Water Extract of Fomitella fraxinea

### Manufacturing Example 1

### Powders

| | |
|---|---|
| powdered water extract of Fomitella fraxinea | 20 mg |
| lactose | 100 mg |
| talc | 10 mg |

The above ingredients were mixed and loaded to a sealed pouch to obtain the formulation in the form of powders.

### Manufacturing Example 2

### Tablets

| | |
|---|---|
| powdered water extract of Fomitella fraxinea | 10 mg |
| corn starch | 100 mg |
| lactose | 100 mg |
| magnesium stearate | 2 mg |

The above ingredients were mixed and tableted according to a conventional tableting method to obtain the formulation in the form of a tablet.

### Manufacturing Example 3

### Capsules

| | |
|---|---|
| powdered water extract of Fomitella fraxinea | 10 mg |
| crystalline cellulose | 3 mg |
| lactose | 14.8 mg |
| magnesium stearate | 0.2 mg |

The above ingredients were mixed and filled into a gelatin capsule according to a conventional capsule manufacturing method to obtain the formulation in the form of a capsule.

### Manufacturing Example 4

### Liquid

| | |
|---|---|
| powdered water extract of Fomitella fraxinea | 20 mg |
| isomerose | 10 g |
| mannitol | 5 g |
| purified water | adequate |

According to a conventional liquid manufacturing method, each of the above ingredients was dissolved by adding purified water, added with an appropriate amount of a lemon flavor, mixed together, added with purified water to a final volume of 100 mL, filled into a brown bottle, and then sterilized to obtain the formulation in the form of a liquid.

### Manufacturing Example 5

### Health Foods

| powdered water extract of Fomitella fraxinea | 20 | mg |
|---|---|---|
| vitamin mixture | adequate | |
| vitamin A acetate | 70 | μg |
| vitamin E | 1.0 | mg |
| vitamin B1 | 0.13 | mg |
| vitamin B2 | 0.15 | mg |
| vitamin B6 | 0.5 | mg |
| vitamin B12 | 0.2 | μg |
| vitamin C | 10 | mg |
| biotin | 10 | μg |
| nicotinic acid amide | 1.7 | mg |
| folic acid | 50 | μg |
| calcium pantothenate | 0.5 | mg |
| mixture of minerals | adequate | |
| ferrous sulfate | 1.75 | mg |
| zinc oxide | 0.82 | mg |
| magnesium carbonate | 25.3 | mg |
| potassium phosphate, monobasic | 15 | mg |
| calcium phosphate, dibasic | 55 | mg |
| potassium citrate | 90 | mg |
| calcium carbonate | 100 | mg |
| magnesium chloride | 24.8 | mg |

Although the mixed ratios of the vitamins and the mixture of minerals disclosed above preferred ones composed of ingredients relatively suitable for manufacturing health foods, various modifications or changes in the mixing ratios may be possible. The above ingredients may be mixed according to the conventional method of manufacturing health foods to be manufactured in the form of granules, and used for manufacturing health food compositions according to the conventional methods.

### INDUSTRIAL APPLICABILITY

Although the preferred embodiment(s) of the present invention have(has) been disclosed for illustrative purposes, those skilled in the art will appreciate that various modifications, additions and substitutions are possible, without departing from the scope and spirit of the invention as disclosed in the accompanying claims.

1. A composition comprising a water extract of Fomitella fraxinea as an active ingredient for treating or preventing obesity.

2. The composition of claim 1, wherein the water extract of Fomitella fraxinea is obtained by extracting Fomitella fraxinea with water at a temperature of from 20 to 60° C. for from 12 to 36 hours.

3. The composition of claim 1, wherein the water extract of Fomitella fraxinea is obtained using from 0.1 to 20 g of Fomitella fraxinea per 100 mL of water.

4. The composition of claim 1, wherein the water extract of Fomitella fraxinea is contained from 0.001 to 50 wt %.

5. The composition claim 1, wherein the extract inhibits the activity of gastrointestinal tract lipase.

6. The composition of claim 5, wherein the gastrointestinal tract lipase is a pancreatic lipase.

7. The composition of claim 1, wherein the extract inhibits the absorption of cholesterol.

8. A functional health food comprising a water extract of Fomitella fraxinea as an active ingredient for improving or preventing obesity.

9. The functional health food of claim 7, wherein the functional health food is a food, a food additive or a drink.

10. The functional health food of claim 8, wherein the functional health food deteriorates the activity of pancreatic cholesterol esterase or gastrointestinal tract lipase or both.

11. The functional health food of claim 10, wherein gastrointestinal tract lipase is pancreatic lipase.

12. A water extract of Fomitella fraxinea, which inhibits the activity of pancreatic cholesterol esterase or gastrointestinal tract lipase or both.

13. The water extract of Fomitella fraxinea of claim 12, which gastrointestinal tract lipase is pancreatic lipase.

14. The water extract of Fomitella fraxinea of claim 12, wherein the water extract of Fomitella fraxinea is obtained by extracting Fomitella fraxinea with water at a temperature of from 20 to 60° C. for from 12 to 36 hours.
